(19) United States
(12) Patent Application Publication
(10) Pub. No.: US 2006/0135957 A1
(43) Pub. Date: Jun. 22, 2006

(54) Method and apparatus to align a probe with a cornea

(76) Inventor: Dorin Panescu, San Jose, CA (US)

Correspondence Address: IRELL & MANELLA LLP, 840 NEWPORT CENTER DRIVE, SUITE 400, NEWPORT BEACH, CA, 92660, US

(21) Appl. No.: 11/021,244

(22) Filed: Dec. 21, 2004

(51) Int. Cl. A61B 18/18

(52) U.S. Cl. 606/41

(57) ABSTRACT

A system and method for denaturing corneal tissue of a cornea. The system may include an optical recognition system that-can recognize a feature of the corneal. The recognized feature is used to register a desired probe location relative to the cornea. The desired probe location is displayed by a monitor. The system further includes a probe that is coupled to an arm. The arm contains position sensors that provide position information of the probe. The position information is used to map and display the actual position of the probe. By watching the monitor the user can move the probe into the desired probe location relative to the cornea. Once the probe is properly positioned energy is delivered to denature corneal tissue.

### BACKGROUND OF THE INVENTION

1. Field of the Invention

The present invention relates to a method and apparatus for treating ocular tissue.

2. Prior Art

Techniques for correcting vision have included reshaping the cornea of the eye. For example, myopic conditions can be corrected by cutting a number of small incisions in the corneal membrane. The incisions allow the corneal membrane to relax and increase the radius of the cornea. The incisions are typically created with either a laser or a precision knife. The procedure for creating incisions to correct myopic defects is commonly referred to as radial keratotomy and is well known in the art.

Radial keratotomy techniques generally make incisions that penetrate approximately 95% of the cornea. Penetrating the cornea to such a depth increases the risk of puncturing the Descemets membrane and the endothelium layer, and creating permanent damage to the eye. Additionally, light entering the cornea at the incision sight is refracted by the incision scar and produces a glaring effect in the visual field. The glare effect of the scar produces impaired night vision for the patient.

The techniques of radial keratotomy are only effective in correcting myopia. Radial keratotomy cannot be used to correct an eye condition such as hyperopia. Additionally, keratotomy has limited use in reducing or correcting an astigmatism. The cornea of a patient with hyperopia is relatively flat (large spherical radius). A flat cornea creates a lens system which does not correctly focus the viewed image onto the retina of the eye. Hyperopia can be corrected by reshaping the eye to decrease the spherical radius of the cornea. It has been found that hyperopia can be corrected by heating and denaturing local regions of the cornea. The denatured tissue contracts and changes the shape of the cornea and corrects the optical characteristics of the eye. The procedure of heating the corneal membrane to correct a patient's vision is commonly referred to as thermokeratoplasty.

U.S. Pat. No. 4,461,294 issued to Baron; U.S. Pat. No. 4,976,709 issued to Sand and PCT Publication WO 90/12618, all disclose thermokeratoplasty techniques which utilize a laser to heat the cornea. The energy of the laser generates localized heat within the corneal stroma through photonic absorption. The heated areas of the stroma then shrink to change the shape of the eye.

Although effective in reshaping the eye, the laser based systems of the Baron, Sand and PCT references are relatively expensive to produce, have a non-uniform thermal conduction profile, are not self limiting, are susceptible to providing too much heat to the eye, may induce astigmatism and produce excessive adjacent tissue damage, and require long term stabilization of the eye. Expensive laser systems increase the cost of the procedure and are economically impractical to gain widespread market acceptance and use.

Additionally, laser thermokeratoplasty techniques non-uniformly shrink the stroma without shrinking the Bowmans layer. Shrinking the stroma without a corresponding shrinkage of the Bowmans layer, creates a mechanical strain in the cornea. The mechanical strain may produce an undesirable reshaping of the cornea and probable regression of the visual acuity correction as the corneal lesion heals. Laser techniques may also perforate Bowmans layer and leave a leucoma within the visual field of the eye.

U.S. Pat. Nos. 4,326,529 and 4,381,007 issued to Doss et al, disclose electrodes that are used to heat large areas of the cornea to correct for myopia. The electrode is located within a sleeve that suspends the electrode tip from the surface of the eye. An isotropic saline solution is irrigated through the electrode and aspirated through a channel formed between the outer surface of the electrode and the inner surface of the sleeve. The saline solution provides an electrically conductive medium between the electrode and the corneal membrane. The current from the electrode heats the outer layers of the cornea. Heating the outer eye tissue causes the cornea to shrink into a new radial shape. The saline solution also functions as a coolant which cools the outer epithelium layer.

The saline solution of the Doss device spreads the current of the electrode over a relatively large area of the cornea. Consequently, thermokeratoplasty techniques using the Doss device are limited to reshaped corneas with relatively large and undesirable denatured areas within the visual axis of the eye. The electrode device of the Doss system is also relatively complex and cumbersome to use.

“A Technique for the Selective Heating of Corneal Stroma” Doss et al., Contact & Intraoccular Lens Medical Jrl., Vol. 6, No. 1, pp. 13-17, January-March, 1980, discusses a procedure wherein the circulating saline electrode (CSE) of the Doss patent was used to heat a pig cornea. The electrode provided 30 volts r.m.s. for 4 seconds. The results showed that the stroma was heated to 70° C. and the Bowman's membrane was heated 45° C., a temperature below the 50-55° C. required to shrink the cornea without regression.

“The Need For Prompt Prospective Investigation” McDonnell, Refractive & Corneal Surgery, Vol. 5, January/February, 1989 discusses the merits of corneal reshaping by thermokeratoplasty techniques. The article discusses a procedure wherein a stromal collagen was heated by radio frequency waves to correct for a keratoconus condition. As the article reports, the patient had an initial profound flattening of the eye followed by significant regression within weeks of the procedure.

“Regression of Effect Following Radial Thermokeratoplasty in Humans” Feldman et al., Refractive and Corneal Surgery, Vol. 5, September/October, 1989, discusses another thermokeratoplasty technique for correcting hyperopia. Feldman inserted a probe into four different locations of the cornea. The probe was heated to 600° C. and was inserted into the cornea for 0.3 seconds. Like the procedure discussed in the McDonnell article, the Feldman technique initially reduced hyperopia, but the patients had a significant regression within 9 months of the procedure.

Refractec, Inc. of Irvine Calif., the assignee of the present application, has developed a system to correct hyperopia with a thermokeratoplasty probe that is connected to a console. The probe includes a tip that is inserted into the stroma layer of a cornea. Electrical current provided by the console flows through the eye to denature the collagen tissue within the stroma. The denatured tissue will change the refractive characteristics of the eye. The process of inserting the probe tip and applying electrical current can be repeated in a circular pattern about the cornea. The pattern may be at 6, 7 and/or 8 millimeters about the center of the cornea. The procedure is taught by Refractec under the service marks CONDUCTIVE KERATOPLASTY and CK.

The spots where the probe is inserted into the cornea are typically marked with a corneal marker. The corneal marker may be a hand held piece that applies an ink ring on the cornea. Manually marking the spots can result in errors. The errors are typically caused by not properly placing the ring about the center of the cornea. A non-concentric ring will result in a non-concentric pattern of denatured spots. A non-concentric pattern of spots could degrade the effectiveness of the procedure and may introduce astigmatism. It would be desirable to provide a system that can more accurately locate the probe relative to the cornea.

### BRIEF SUMMARY OF THE INVENTION

A system and method for denaturing corneal tissue of a cornea. The method includes recognizing a feature of the cornea, displaying a desired probe location based on the recognized feature and moving a probe to the desired location to deliver energy and denature corneal tissue.

### BRIEF DESCRIPTION OF THE DRAWINGS

FIG. 1 is a perspective view of a system for denaturing corneal tissue;

FIG. 2 is a schematic of a controller of the system;

FIG. 3 is a front view of a monitor that displays a desired probe location and an actual probe location;

FIG. 4 is top view showing a pattern of denatured spots in a cornea;

FIG. 5 is a graph showing a waveform that is provided by a console of the system.

### DETAILED DESCRIPTION

Disclosed is a system and method for denaturing corneal tissue of a cornea. The system-may include an optical recognition system that can recognize a feature of the corneal. The recognized feature is used to register a desired probe location relative to the cornea. The desired probe location is displayed by a monitor. The system further includes a probe that is coupled to an arm. The arm contains position sensors that provide position information of the probe. The position information is used to map and display the actual position of the probe. By watching the monitor the user can move the probe into the desired probe location relative to the cornea. Once the probe is properly positioned energy is delivered to denature corneal tissue.

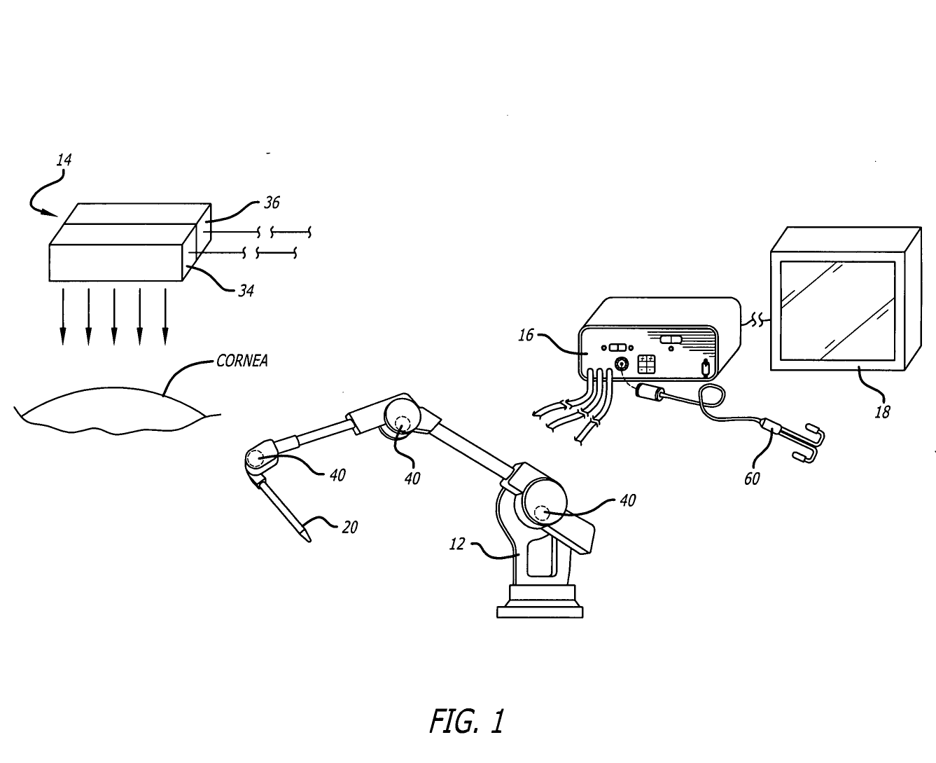

Referring to the drawings more particularly by reference numbers, FIG. 1 shows an embodiment of a system 10 that can be used to denature corneal tissue. The system 10 includes an arm 12 and an optical detector 14 that are connected to a controller 16. The controller 16 is connected to a display monitor 18. The arm 12 holds a probe 20 that can deliver energy and denature corneal tissue. Although a monitor 18 is shown and described, the system 10 may utilize another device such as a head-up display (not shown) that is worn by the user and displays the desired and actual positions of the probe. As another embodiment, the system 10 may also have a printer that prints out the desired and actual probe locations.

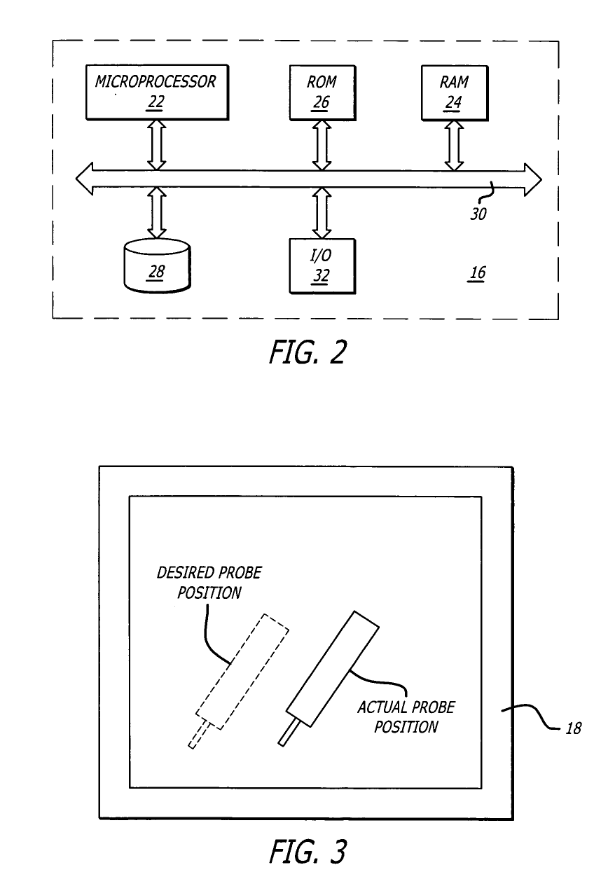

As shown in FIG. 2 the controller 16 may include at least one microprocessor 22, volatile memory (RAM) 24, non-volatile memory (ROM) 26 and a mass storage device (HDD) 28 all connected to a bus 30. The controller 18 may have I/O ports 32 with the associated driver circuit, A/D, D/A, etc. for interfacing with the optical detector 14, the arm 12, the monitor 18 and the probe. The processor 22 may perform operations in accordance with data and instructions provided by software/firmware.

Referring to FIG. 1, the optical detector 14 may include a light source 34 and a photodetector 36. The light source 34 may be an array of light emitting diodes (LEDs). The photodetector 36 may be a camera. The camera provides input signals to the controller 16. The controller 16 may contain a software/firmware routine that can recognize a feature of the cornea. For example, the optical detector may recognize a pupil of the cornea. The pupil may be recognized in accordance with the techniques described in the articles “A Method for Size Estimation of Amorphous Pupil in 3-Dimensional Geometry”, by Jieun Kim et al., Sep. 1-5, 2004, IEEE and “An algorithm to Detect a Center of Pupil for Extraction of Point of Gaze”, S. I. Kim et al., Sep. 1-5, 2004 IEEE, which are hereby incorporated by reference.

The arm 12 may include a number of linkages joined by various joints to provide multiple degrees of freedom. The multiple degrees of freedom allow a user to move the probe 20 to any location of a cornea. Each joint includes a position sensor 40 that provides 3-D coordinates position information to the controller 16. The controller 16 contains software/firmware that maps the position of the probe relative to the cornea based on the position information. The arm and mapping software may be a system sold by Immersion of San Jose, Calif. under the product name MicroScribe. Although an arm 12 is shown and described, it is to be understood that the position of the probe 20 can be provided by other devices such as a wireless transmitter that provides free space position information. For example, the system may include transmitters and receivers that can locate the position of the probe 20 through triangulation or other known methods.

The system 10 can be calibrated by using a cornea feature, such as the center of the pupil, to provide a reference datum for the mapping software. This can be done by initially identifying the center of the pupil and displaying the center on the monitor. The center may be displayed graphically. The actual position of the probe is also depicted by the monitor so that the user can use the monitor to accurately place the probe at the center of the pupil. The user may then provide an input such as depressing a button that is interpreted by the controller 16 to register that point on the cornea as the reference datum. The user may repeat the process for a point on the cornea outward for the center. The second data point can be used to register the probe in a radial coordinate system.

Once the reference datum has been identified the controller 16 can then register a desired location of the probe for denaturing tissue. The desired location can be represented graphically by the monitor as shown in phantom in FIG. 3. To facilitate precise placement of probe 20, the optical detector 14 can shine light, using the LEDs 34, such that it optically marks the desired locations of the denatured areas 50. Although the preferred embodiment presents an optical marking system, other marking systems could be implemented by the skilled in the art without departing from the broad concept of this invention. For example, an ink marker could be used to mark the desired locations of the denatured areas 50. Then, the operator registers the ink marks with respect to the reference system provide by the arm 12 and the controller 16. As presented above for the optical marker, the registration process may involve aligning the center of the marker with the center of the pupil and then defining at least one more data point for radial alignment (e.g. place the probe 20 over one of the ink marks). Once the registration and marking steps are completed, the probe placement process commences. The actual probe location is also displayed as shown in FIG. 3. The user can move the probe and watch the monitor to accurately locate the probe relative to the cornea. By way of example, the probe can be considered properly located when the actual probe overlaps the desired probe location on the display 18. Although a phantom probe is shown it is to be understood that the desired probe location can be displayed in a variety of ways. For example, the monitor 18 may sow a video image of the cornea while displaying a graphical area of the desired probe locations.

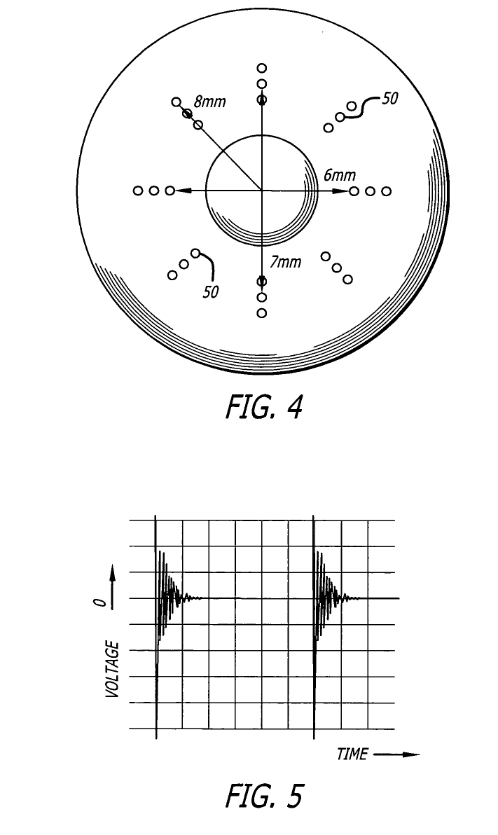

FIG. 4 shows a pattern of denatured areas 50 that have been found to correct hyperopic or presbyopic conditions. A circle of 8, 16, or 24 denatured areas 50 are created about the center of the cornea, outside the visual axis portion of the eye. The visual axis typically has a nominal diameter of approximately 5 millimeters. It has been found that 16 denatured areas provide the most corneal shrinkage and less post-op astigmatism effects from the procedure. The circles of denatured areas typically have a diameter between 6-8 mm, with a preferred diameter of approximately 7 mm. If the first circle does not correct the eye deficiency, the same pattern may be repeated, or another pattern of 8 denatured areas may be created within a circle having a diameter of approximately 6.0-6.5 mm either in line or overlapping. The assignee of the present application provides instructional services to educate those performing such procedures under the service marks CONDUCTIVE KERATOPLASTY and CK.

The exact diameter of the pattern may vary from patient to patient, it being understood that the denatured spots should preferably be formed in the non-visionary portion of the eye. Although a circular pattern is shown, it is to be understood that the denatured areas may be located in any location and in any pattern. In addition to correcting for hyperopia, the present invention may be used to correct astigmatic conditions. For correcting astigmatic conditions, the denatured areas are typically created at the end of the astigmatic flat axis. The technique may also be used to correct procedures that have overcorrected for a myopic condition.

Once the center of the pupil is identified the controller 16 can register a desired probe location. The controller 16 can create desired probe locations to correlate with the mid-peripheral locations of the cornea such as the 6, 7 and/or 8 diameter circles shown in FIG. 4. The controller 16 and monitor 18 may display a plurality of desired probe locations about a real or graphical depiction of the cornea. The display of each probe location may disappear after the creation of a denatured spot at the corresponding cornea location.

The probe 20 may be the same or similar to a device sold by the assignee of this application, Refractec of Irvine, California. The probe may include an electrode that is inserted into the cornea to deliver radio frequency electrical energy. Alternatively, the probe 20 may transmit energy to denature the cornea without direct contact with the cornea (e.g. probe 20 can be a laser diode). The energy generates heat that denatures the corneal tissue. The probe may be a mono-polar device or a bi-polar device. If the probe is a mono-polar device then the system would typically include a ground element 60. The ground element 60 may be integrated into a lid speculum that is used to maintain the eyelids in an open position. The probe 20 may also include a stop that limits the penetration depth of the electrode.

The controller 16 may provide a predetermined amount of energy to the probe 20, through a controlled application of power for a predetermined time duration. The controller 16 may have manual controls that allow the user to select treatment parameters such as the power and time duration. The controller 16 can also be constructed to provide an automated operation. The controller 16 may have monitors and feedback systems for measuring physiologic tissue parameters such as tissue impedance, tissue temperature and other parameters, and adjust the output power of the radio frequency amplifier to accomplish the desired results.

In one embodiment, the controller 16 provides voltage limiting to prevent arcing. To protect the patient from overvoltage or overpower, the controller 16 may have an upper voltage limit and/or upper power limit which terminates power to the probe when the output voltage or power of the unit exceeds a predetermined value.

The controller 16 may also contain monitor and alarm circuits which monitors physiologic tissue parameters such as the resistance or impedance of the load and provides adjustments and/or an alarm when the resistance/impedance value exceeds and/or falls below predefined limits. The adjustment feature may change the voltage, current, and/or power delivered by the controller 16 such that the physiological parameter is maintained within a certain range. The alarm may provide either an audio and/or visual indication to the user that the resistance/impedance value has exceeded the outer predefined limits. Additionally, the unit may contain a ground fault indicator, and/or a tissue temperature monitor. The front panel of the controller 16 typically contains meters and displays that provide an indication of the power, frequency, etc., of the power delivered to the probe.

The controller 16 may deliver a radiofrequency (RF) power output in a frequency range of 100 KHz-5 MHz. In the preferred embodiment, power is provided to the probe at a frequency in the range of 350 KHz. The time duration of each application of power to a particular location of tissue can be up to several seconds.

If the system incorporates temperature sensors, the controller 16 may control the power such that the target tissue temperature is maintained to no more than approximately 100° C., to avoid necrosis of the tissue. The temperature sensors can be carried by the probe 20, incorporated into the probe electrodes, or attached within proximity of the electrodes.

If the system includes an impedance monitor, the power could be adjusted so that the target tissue impedance, assuming a probe 20 with a tip of length 460 μm and diameter of 90 μm, decreases by approximately 50% from an initial value that is expected to range between 1100 to 1800 ohm. If two or more electrodes are energized in parallel, the initial impedance values may be less than 1000 ohm. For bipolar applications, the initial impedance values may be higher, over 2000 ohms, under nominal circumstances. The controller 16 could regulate the power down if, after an initial descent, the impedance begins to increase.

Controls can be incorporated to terminate RF delivery if the impedance increases by a significant percentage from the baseline. Alternatively, or additionally, the controller 16 could modulate the duration of RF delivery such that delivery is terminated only when the impedance exceeds a preset percentage or amount from a baseline value, unless an upper time limit is exceeded. Other time-modulation techniques, such as monitoring the derivative of the impedance, could be employed. Time-modulation could be based on physiologic parameters other than tissue impedance (e.g tissue water content, chemical composition, etc.)

FIG. 5 shows a typical voltage waveform that is delivered by the probe 20 to the skin. Each pulse of energy delivered by the probe 20 may be a highly damped sinusoidal waveform, typically having a crest factor (peak voltage/RMS voltage) greater than 5:1. Each highly damped sinusoidal waveform is repeated at a repetitive rate. The repetitive rate may range between 4-12 KHz and is preferably set at 7.5 KHz. Although a damped waveform is shown and described, other waveforms, such as continuous sinusoidal, amplitude, frequency or phase-modulated sinusoidal, etc. can be employed.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

For example, although the delivery of radio frequency energy is described, it is to be understood that other types of non-thermal energy such as direct current (DC), microwave, ultrasonic and light can be transferred into the cornea. Non-thermal energy does not include the concept of heating a tip that had been inserted or is to be inserted into the cornea.

By way of example, the controller 16 can be modified to supply energy in the microwave frequency range or the ultrasonic frequency range. By way of example, the probe 20 may have a helical microwave antenna with a diameter suitable for corneal delivery. The delivery of microwave energy could be achieved with or without corneal penetration, depending on the design of the antenna. The system may modulate the microwave energy in response to changes in the characteristic impedance.

For ultrasonic application, the probe 20 would contain a ultrasonic transducer that oscillates a tip. The system could monitor acoustic impedance and provide a corresponding feedback/regulation scheme. For application of light the probe may contain some type of light guide that is inserted into the cornea and directs light into corneal tissue. The console would have means to generate light, preferably a coherent light source such as a laser, that can be delivered by the probe. The probe may include lens, waveguide and a photodiode that is used sense reflected light and monitor variations in the index of refraction, birefringence index of the cornea tissue as a way to monitor physiological changes and regulate power.

Although one controller 16 is shown and described, it is to be understood that the system may contain multiple controllers. For example, the system may have one controller to map probe locations and another controller to deliver energy to the probe 20.

Although an arm 12 is presented to implement the mapping to the probe 20 locations, other devices could be used by those skilled in the art to achieve semi-automated probe placement. Robotic actuators driven, for example, interactively by joystick motion could be another implementation. Such variation to the presented preferred embodiment should not alter the broad concept of the invention, which is to position an energy-delivery probe at mapped corneal features semi-automatically, in an interactive fashion.

What is claimed is:

1. An ophthalmic system used to denature corneal tissue of a cornea, comprising:
a probe adapted to provide energy to the corneal tissue;
a position sensor that is coupled to and can sense a position of said probe;
a location device that provides the desired location of said probe and an actual position of said probe relative to the cornea; and,
a controller that is coupled to said position sensor, said controller references the actual position with the desired position and causes said location device to provide the desired and actual positions.

2. The system of claim 1, wherein said device includes a display monitor.

3. The system of claim 1, wherein said position includes an arm with a plurality of position sensors.

4. The system of claim 1, wherein said controller utilizes a feature of the cornea as a reference datum in referencing the desired and actual locations of said probe.

5. The system of claim 4, wherein said probe is placed on the feature of the cornea to create the reference datum.

6. The system of claim 1, wherein said controller and said device display a plurality of desired probe locations.

7. The system of claim 1, wherein said probe includes an electrode with a stop that limits a penetration depth of said electrode into the cornea.

8. The system of claim 1, wherein said controller provides energy to said probe to denature the corneal tissue.

9. The system of claim 8, wherein said energy is electrical energy.

10. The system of claim 8, wherein said energy is in a microwave frequency.

11. The system of claim 8, wherein said energy is optical.

12. The system of claim 8, wherein said energy is ultrasound.

13. The system of claim 8, wherein said probe includes at least two electrodes.

14. The system of claim 1, further comprising a photodetector that is coupled to said controller to image the cornea.

15. The system of claim 1, further comprising a marking system to mark the cornea and said controller registers the desired location from the mark.

16. The system of claim 1, wherein said controller maps the desired location.

17. An ophthalmic system used to denature corneal tissue of a cornea, comprising:
a probe adapted to provide energy to the corneal tissue;
sensor means for sensing an actual position of said probe;
controller means for referencing a desired location with the actual location of said probe relative to the cornea; and,
location means for providing the desired location of said probe and an actual position of said probe relative to the cornea.

18. The system of claim 17, wherein said location means includes a monitor.

19. The system of claim 17, wherein said sensor means includes an arm with a plurality of position sensors.

20. The system of claim 17, wherein said controller means utilizes a feature of the cornea as a reference point for referencing the desired and actual locations of said probe.

21. The system of claim 20, wherein said probe is placed on the feature of the cornea to create the reference point.

22. The system of claim 17, wherein said controller means and said monitor display a plurality of desired probe locations.

23. The system of claim 17, wherein said probe includes a stop that limits a penetration depth of said probe into the cornea.

24. The system of claim 17, wherein said controller means provides energy to said probe to denature the corneal tissue.

25. The system of claim 24, wherein said energy is electrical energy.

26. The system of claim 24, wherein said energy is in a microwave frequency.

27. The system of claim 24, wherein said energy is optical.

28. The system of claim 24, wherein said energy is ultrasound.

29. The system of claim 17, further comprising imaging means for imaging the cornea.

30. The system of claim 17, further comprising marking means for marking the cornea and said controller means registers the desired location from the mark.

31. The system of claim 17, wherein said controller means maps the desired location.

32. A method for denaturing a cornea, comprising:
providing a probe that can deliver energy;
referencing a desired location with an actual location of the probe;
displaying a desired location and an actual location of a probe relative to the cornea; and,
moving the probe to the desired location and delivering energy to denature corneal tissue.

33. The method of claim 32, further comprising recognizing a feature of the cornea to reference the desired and actual locations of-the probe.

34. The method of claim 33, wherein the probe is placed at the-feature of the cornea to create a reference point for referencing the desired and actual probe locations.

35. The method of claim 32, wherein the probe is moved with an arm that provides positional information.

36. The method of claim 32, wherein the probe is inserted into the cornea.
